(19) United States
(12) Patent Application Publication
(10) Pub. No.: US 2014/0316421 A1
(43) Pub. Date: Oct. 23, 2014

(54) METHOD AND TOOL WITH INTEGRATED INCLINOMETER

(71) Applicant: RL INVENTIONS, LLC, Miami, FL (US)

(72) Inventor: Lisa Sanders, Miami Beach, FL (US)

(73) Assignee: RL Inventions, LLC, Miami, FL (US)

(21) Appl. No.: 13/868,465

(22) Filed: Apr. 23, 2013

(51) Int. Cl. A61B 17/88; A61B 19/00

(52) U.S. Cl. CPC A61B 17/8883; A61B 19/46
USPC 606/102

(57) ABSTRACT

A tool includes a wrench, an inclinometer integrated with the wrench, and an electronic indicator operatively coupled to the inclinometer. The indicator indicates when a predetermined rotation is met or indicates when a contrary rotation direction is occurring. In another embodiment, a tool can include a shaft for applying a torque, an inclinometer integrated with the shaft, a memory coupled to the inclinometer and a processor communicatively coupled to the memory. The processor performs operations of generating a signal indicating when a predetermined rotation is met or when a contrary rotation direction is occurring. A method of operating the tool can include rotating a member of the tool, indicating when a predetermined rotation is met by rotating the member, and indicating when a contrary rotational direction is occurring by rotating the member in a counter-indicated direction. Additional embodiments are disclosed.

### CROSS-REFERENCE TO RELATED APPLICATIONS

N/A.

### FIELD

The embodiments relate to a method and tool having an integrated inclinometer that provides an indication of appropriate rotation.

### BACKGROUND

Distraction osteogenesis is a surgical process used to reconstruct skeletal deformities and lengthen the long bones of the body. A corticotomy is used to fracture the bone into two segments, and the two hone ends of the bone are gradually moved, apart during the distraction phase, allowing new bone to form in the gap. When the desired or possible length is reached, a consolidation phase follows in which the bone is allowed to keep healing. Distraction osteogenesis has the benefit of simultaneously increasing bone length and the volume of surrounding soft tissues.

The most common technique is the Ilizarov surgery with the Ilizarov external fixator. Ilizarov surgery, developed by Gavriil Ilizarov, a Russian orthopedic surgeon, in 1951, is the oldest and most common method of distraction osteogenesis. It often brings complications. The process involves shattering bones and devascularised others that are removed from the patient, leaving a gap. A healthy part of an upper bone can be broken into two segments with an external saw and then the leg is fitted with the Ilizarov frame that pierces through the skin, muscles, and bone. Screws attached to the middle bone are turned 1 millimetre (mm) per day, so that new bone tissues that are formed in the growth zone are gradually pulled apart to increase the gap (one millimetre has been found to be the optimal bone distraction rate. Lengthening too fast overstretches the soft tissues, resulting not only in pain, but also in the inability of the bone to fill up the gap; too slow, and the bone hardens before the full lengthening process is complete. After the gap is closed, the patient continues to wear the frame until the new bone solidifies. The waiting period before the frame can be removed is usually one month per centimeter of lengthened bone.

Ilizarov surgery is extremely painful, uncomfortable, infection-prone, and often causes unsightly scars. Frames used to be made of stainless steel rings weighing up to 7 kilogram (kg), but newer models are made of carbon fiber reinforced plastic, which though lighter, are equally cumbersome.

Derivative devices provide physicians better control over the bone axis angle during elongation, such as the Taylor Spatial Frame (TSF) which is computer assisted. The downside of these developments are their relative complexity and resulting longer learning curve.

For decades, the Ilizarov procedure was the best chance for shattered bones to be restored, and crooked ones shattered. Breakthroughs in distraction osteogenesis in the 1990s, however, have resulted in less painful (albeit more expensive) alternatives, such as unilateral rails. In any case, tools are used to rotate the attached hardware and great care should be used when manipulating such hardware in the distraction osteogenesis process.

### SUMMARY

One embodiment of the present disclosure can entail tool including a wrench, an inclinometer integrated with the wrench, and an indicator operatively coupled to the inclinometer, indicating when a predetermined rotation is met or indicating when a contrary rotation direction is occurring.

Another embodiment of the present disclosure can a tool including a shaft for applying torque, an inclinometer integrated with the shaft, as memory storing computer instructions, the memory coupled to the inclinometer, and a processor communicatively coupled to the memory. The processor, responsive to executing the computer instructions, performs operations including generating a signal indicating when a predetermined rotation is met or generating a signal indicating when is contrary rotation direction is occurring.

Yet another embodiment of present disclosure can entail a method of operating a tool by rotating a member of the tool, the tool having an inclinometer integrated with the member, indicating when a predetermined rotation is met by rotating the member, and indicating when a contrary rotational direction is occurring by rotating the member in a counter-indicated direction.

### BRIEF DESCRIPTION OF THE DRAWINGS

FIG. 1 is a representation of a tool having an integrated inclinometer in accordance with the embodiments;

FIG. 2 illustrates the tool of FIG. 1 rotated at a 45 degree angle from a zeroed position;

FIG. 3 illustrates the tool of FIG. 1 rotated at a 90 degree angle from a zeroed position;

FIG. 4 is a diagram illustrating an alternative tool similar to the tool of FIG. 1 in accordance with the embodiments herein;

FIG. 5 illustrates the alternative tool of FIG, 4 rotated in a counter indicated direction in accordance with the embodiments;

FIG. 6 illustrates a diagram of yet another alternative tool rotated in an appropriate direction in accordance with the embodiments;

FIG. 7 illustrates the tool of FIG. 6 rotated in an counter-indicated direction in accordance with the embodiments;

FIG. 8 illustrates a diagram of yet another alternative tool rotated in an appropriate direction in accordance with the embodiments;

FIG. 9 illustrates the tool of FIG. 8 rotated in an counter-indicated direction in accordance with the embodiments;

FIG. 10 illustrates a diagram of yet another alternative tool set in as zeroed position in accordance with the embodiments;

FIG. 11 illustrates the alternative tool of FIG. 10 dialed-in for a 45 degree rotation of the tool in accordance with the embodiments;

FIG. 12 illustrates the alternative tool of FIG. 11 after the tool has rotated 45 degrees in accordance with the embodiments;

FIG. 13 is a flow chart illustrating a method of operating a tool in accordance with the embodiments;

FIG. 14 is a diagram of an computing system used in conjunction with the tools and methods herein in accordance with the embodiments.

### DETAILED DESCRIPTION

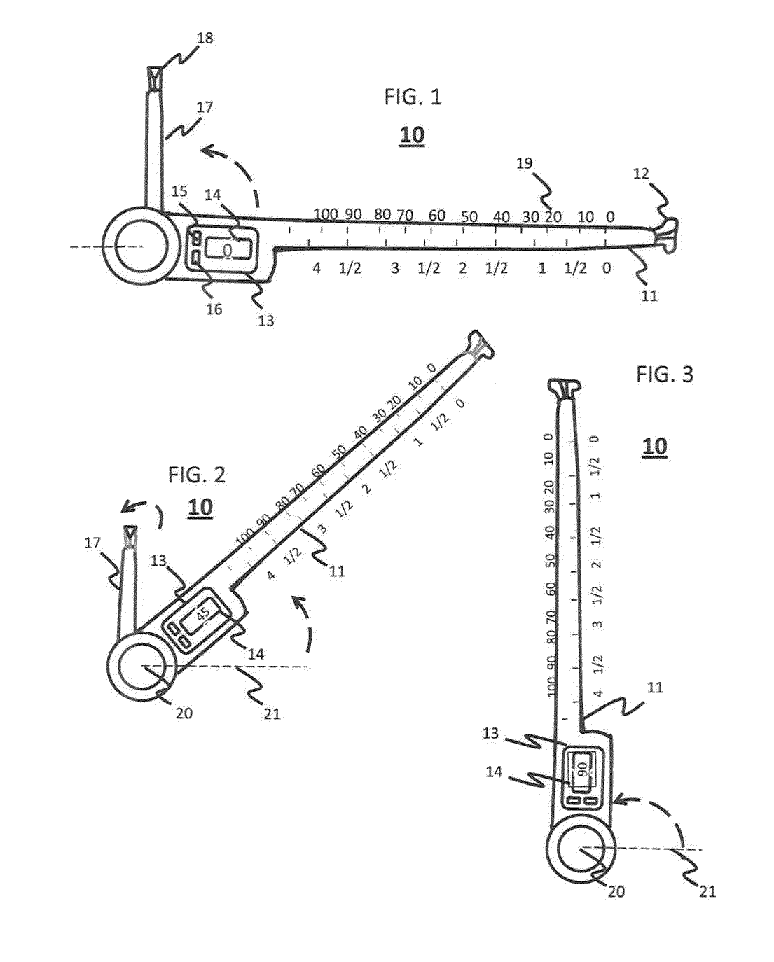

FIG. 1 is a diagram of a tool 10 having an integrated inclinometer 13. The tool 10 can be a wrench, but the embodiments are not limited to a wrench or even to a particular type of wrench. For example, the tool can be an Allen wrench, a socket wrench, a screwdriver, or even a drill or other types of tools. The tool 10 can include an indicator 14 operatively coupled to the inclinometer 13. indicating when a predetermined rotation is met or indicating when a contrary rotation direction is occurring. Note that a contrary rotation of the tool can result in bone compression and/or disfigurement.

The tool 10 can include a shaft 11 for applying torque where the inclinometer 13 can be integrated with the shaft 11. The tool can further include a memory (see FIG. 11) storing computer instructions where the memory is coupled to the inclinometer and a processor communicatively coupled to the memory. The processor, responsive to executing the computer instructions, performs operations as will be further described below. The inclinometer 13 can include a zeroing function button 15 that sets a starting angle at zero and an on/off function button 16. In the context of an Allen wrench or other types of wrenches, the tool 10 can include a second shaft 17 having a tool head 18. The shaft 11 cart also optionally include a ruler 19 that provides measurements in millimeters on a top side for example and includes measurements in inches for subdivisions of inches) on a bottom side for example. The ruler is not limited to millimeters or inches, but other measurements can be used as needed for a particular application. The shaft 11 can also optionally include a tool head 12 that can be used in an orientation distinct from the shaft 17.

FIG. 2 illustrates the tool 10 being rotated in a counterclockwise direction about a pivot point 20 from a starting line of reference 21. In FIG. 2, the tool 10 is rotated at a 45 degree angle as represented in the display 14 of the inclinometer 13. In FIG. 3, the toot s rotated at a 90 degree angle as represented display 14 of the inclinometer 13.

The indicator 14 noted above can be a digital display operatively coupled to the inclinometer 13 and the tool can include computer instructions when executed by the processor to performs operations of generating the signal indicating when the predetermined rotation is met an generating the signal when a contrary rotation direction is occurring. The indicator not necessarily need to be physically in connection with the tool 10 or inclinometer 13, but can ne a display coupled to a computing device remote from the meter such as a lap top, smart phone, or other remote computing device. The tool 10 can also generate an a audible alert when the predetermined rotation is met or when the contrary rotation direction is occurring. The alert can be heard via a speaker embedded in the tool or in a remote computing device. The tool can further enable a calendaring and reminder alert function for a subsequent predetermined rotation. This can be useful in the context of an Ilizarov or a Taylor spatial frame (TSF) device where subsequent rotations within prescribed time periods are part of the treatment.

In another embodiment, the tool can generate a user interface for programming the predetermined rotation and for programming a schedule for performing a number of the predetermined rotations within a predetermined time frame. In this regard, the tool 10 can include a wireless interface for coupling the processor to a computing device coupled to a display as can be seen in FIG. 11.

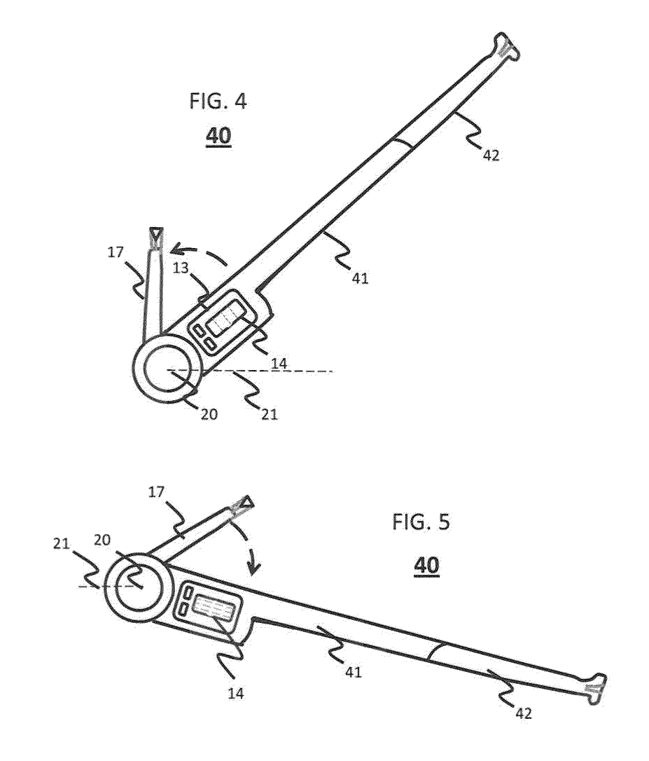

Referring to FIG. 4, the inclinometer 13 of a tool 40 can be a digital inclinometer and the indicator 14 can be a digital display displaying a first display color light as shown in FIG. 4 when a correct rotation direction is occurring and displaying a second display color light when the contrary rotation direction is occurring as illustrated in FIG. 5. The “correct” or appropriate rotation and the “contrary” or “counter-indicated” rotation direction can be programmed into the inclinometer. In this instance, a counter-clockwise rotation can be considered the correct rotation and a clockwise rotation can be considered a contrary or counter-indicated rotation direction. The first display color light for the appropriate rotation can be a green light and the second display color light for a counter indicated or contrary rotation can be a red light such as the light displayed in the indicator 14 of FIG. 5. Further note that the tool 40 can include a shaft formed in two portions, namely shaft portion 41 and shaft portion 42. The inclinometer 13 can be self-powered with a button cell type battery or can alternatively be powered with a battery such as a AAA or AA form-factored battery that can fit within a chamber within either shaft portion 41 or shaft portion 42. The shaft portions can mate with threaded mating portions.

In the context of an Ilizarov or TSF device, the color on the display can be programmed to remain green as long as the current rotation is within a prescribed range in an appropriate rotational direction. For example, a counter-clockwise rotation within 90 degrees from a zeroed position will provide a green light in the display 14 whereas a rotation beyond the 90 degrees from the zeroed position will provide a different color in the display such as a red color. A rotation in the clockwise direction from the zeroed position will also provide the different color indicating that the rotation is in a counter-indicated direction. In one embodiment, a counter-clockwise rotation of the tool can correspond to an appropriate separation in bone portions as prescribed in the Ilizarov technique whereas a clockwise rotation of the tool can correspond to a compression of the bone portions which is not prescribed. The tool can be used to adjust threaded rods of an Ilizarov apparatus or of an TSF device. Since the users (which can include the patient themselves or their caretakers) of such devices can easily get confused as to the appropriate direction and the millimeter movements involved create pain whether bones are being compressed or separated, an additional indicator as provided herein will reduce the likelihood that the tool user will rotate the tool in a manner that is not prescribed. In most instances, the user of the tool is primarily the patient or the caretaker. A physician (also considered a caretaker) can likewise utilize the tool and benefit from many of the features described herein.

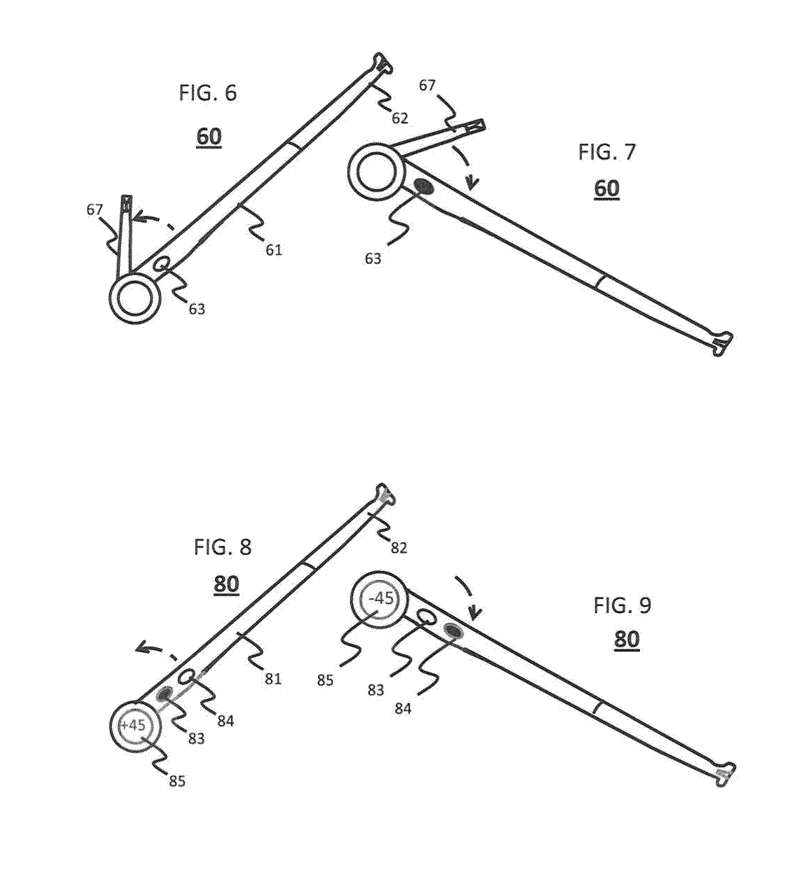

Referring to FIGS. 6 and 1, a similar tool 60 to the tool 40 of FIGS. 4 and 5 includes a shaft having portions 61 and 62 similar to shaft portions 41 and 42 and a second shaft 61 similar to second shaft 17. The shaft or shaft portion 61 can include an indicator 63 that can be at least one light emitting diode that displays a plurality of different color lights. The single light emitting diode can be controlled to display a first color such as green when the tool 60 is rotated within prescribed ranges in an appropriate direction and display a second color such as red when the tool 60 is rotated outside prescribed ranges or in a counter-indicated direction. The light emitting diode or indicator 63 is controlled in response to measurements taken by an embedded inclinometer (not shown within the tool 60. Multiple colors can also be used to indicate varying degrees from prescribed ranges. As illustrated in FIG. 6, the light emitting diode 63 can be emitting a first color for an appropriate rotation and as illustrated in FIG. 7, the light emitting diode 63 can be emitting a second color for a contrary or counter-indicated rotation of the tool 60.

Referring to FIGS. 8 and 9, another similar tool 80 to the tool 60 of FIGS. 6 and 7 includes a shaft having shaft portions 81 and 82. The tool 80 further includes an indicator that can include at least one light emitting diode 83 displaying a first color light and at least a second color diode 84 displaying a second color light. Operationally, the light emitting diode 83 (of the first color) can be active when (the inclinometer of the tool) senses an appropriate rotation as illustrated in FIG. 8 and the light emitting diode 84 can be active when the tool senses a counter-indicated or contrary rotation as shown in FIG. 9. In one embodiment, the indicator can alternatively or additionally include a display 85 operatively coupled to an embedded or integrated inclinometer. The display can be incorporated on the pivot point as shown in FIGS. 8 and 9. A rotation in an appropriate direction can show a positive rotation measurement such as “+45” (see FIG. 8) and a contrary or counter indicated rotation can display a negative rotation measurement such as “−45” (see FIG. 9). As described above, the tool can be an Allen wrench, socket wrench, screwdriver, drill or other tool and can further include one or more rulers. One or more embodiments can include a power source forming a portion of the shaft. Such a power source can power one or more of the inclinometer, any associated processor, or any of the various indicators such as the displays, light emitting diodes, audible alerting devices or other possible indicators. Such a power can power one or more of the devices noted above or such devices can be powered independent of a battery formed in the shaft.

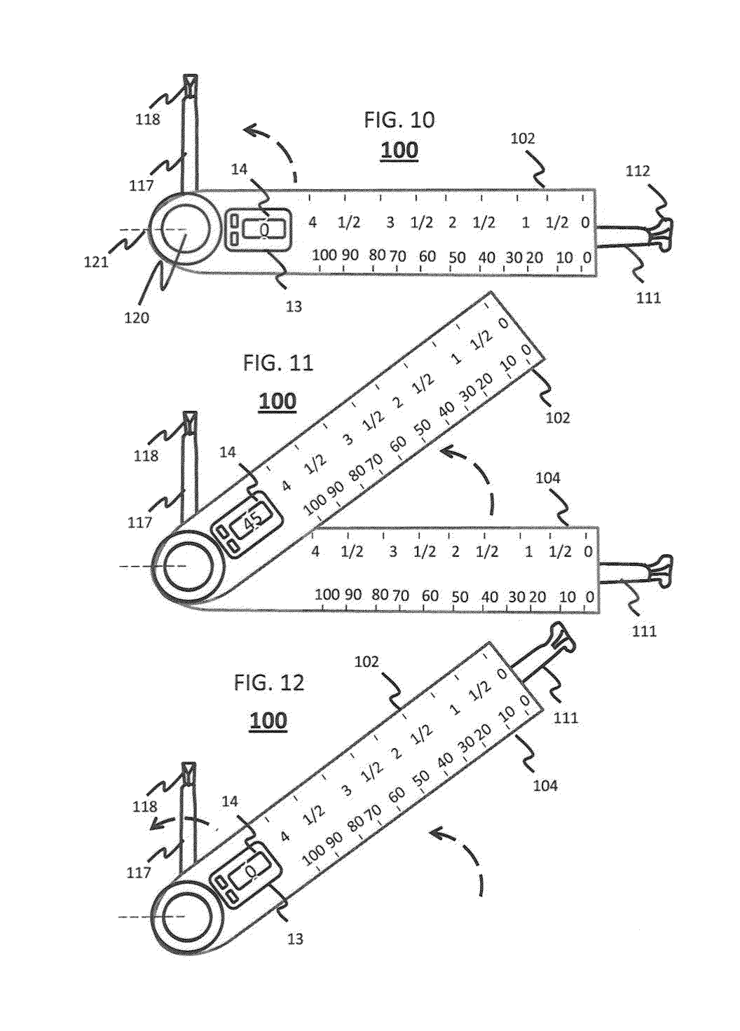

Referring to FIGS. 10-12, a series of diagrams illustrate another embodiment of a tool 100 that includes an inclinometer 13 affixed to a first member 102 (which can be a first ruler) and a wrench having a first shaft 111 and a second shaft 117 affixed to a second member 104 (which can be a second ruler). The second shaft 117 can include a tool head 118. The first shaft can optionally include a tool head 112). The first member 102 can rotate relative to the second member 104. The inclinometer 13 having a display 14 provides a measure of the relative rotation between the first member 102 and the second member 104.

Operationally, when the two members 102 and 104 are flush together as shown in FIG. 10, the inclinometer will read as zero. As shown in FIG. 11, the first member 102. can rotate freely or independently from the second member 104 and the affixed wrench. The first member 102 can be used to “dial-in” a predetermined rotation. For example, if a user wants to rotate the second shaft 117 (and the first shaft 111) a 45 degree angle, the user can rotate the first member 102 having the inclinometer affixed to the first member 102 to a prescribed angle such as 45 degrees first as illustrated on the display 14 of the inclinometer in FIG. 11. The shaft 117 has not rotated at this point since the first (102) and second (104) members rotate independently. Then, the user can move the second member 104 (and affixed shaft 111) until the second member 104 is flush against the first member 102 as shown in FIG. 12 and the inclinometer display 14 reads zero again. In other words, after the user dials-in the desired angle by rotating the first member 102, the second member 104 and affixed wrench can subsequently be rotated “up” to the first member 102 and the inclinometer will count-down from the 45 degrees down to the new zeroed position (as shown in FIG. 12) to effectively and accurately rotate the shaft 117 the desired angle (of 45 degree in this example).

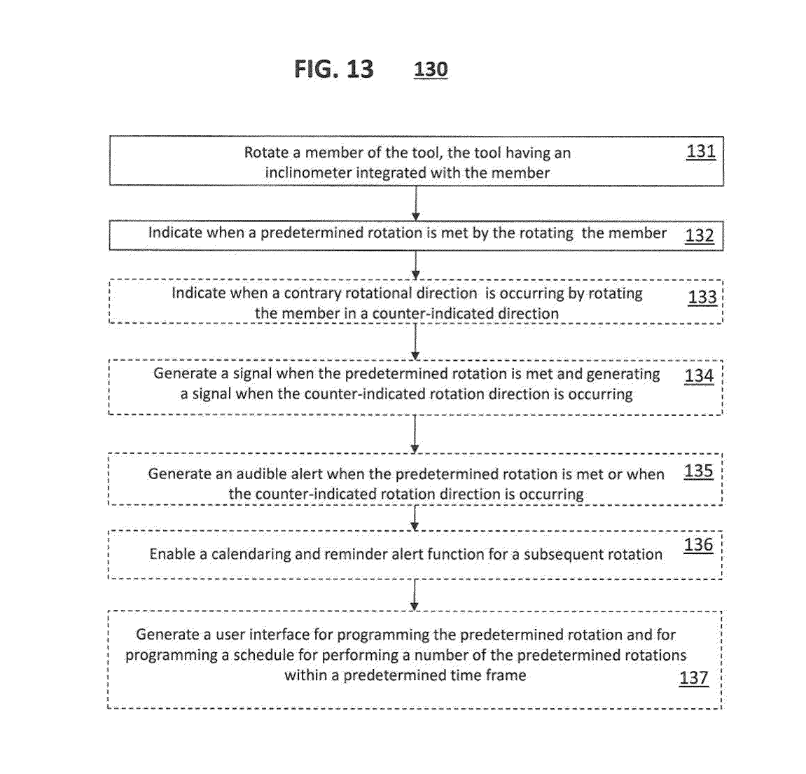

FIG. 13 illustrates a flow chart representing a method 130 of operating a tool as described FIGS. 1-12 above. The method 130 can include rotating a member of the tool at 131 where the tool has an inclinometer integrated with the member. At 132. the method an indicate when a predetermined rotation is met by rotating the member. the method at 133 can further indicate when a contrary rotational direction is occurring by rotating the member in a counter-indicated direction. At 134, the method can generate a signal indicating when the predetermined rotation is met and can also generate a signal when a counter-indicated (or contrary) direction is occurring. Optionally at 135, an audible alert can be generated when the predetermined rotation is met or when the counter-indicate rotation is occurring. In one embodiment, the method can generate a calendaring and reminder alert function for a prescribed subsequent rotation. In yet another embodiment, the method can generate a user interface for programming the predetermined rotation and for programming a schedule for performing a number of the predetermined rotations within a predetermined time frame.

Again, within the context of a Ilizarov or TSF device, a full rotation or 360 degrees can correspond to a 1 millimeter separation of bone portions. In most instances, the 1 millimeter separation is preferably performed over several instances within a day. One possible regimen is a quarter rotation or 90 degree rotation every 6 hours within a day. The treatments can span several months and accuracy in tracking rotation and direction can be implemented in a calendaring and reminder alert function as noted above. The calendaring and reminder alert function can be programmable and the user interface can be used to set any predetermined or proscribed rotation schedule desired to implement the appropriate regimen prescribed by a doctor such as an orthopedic surgeon.

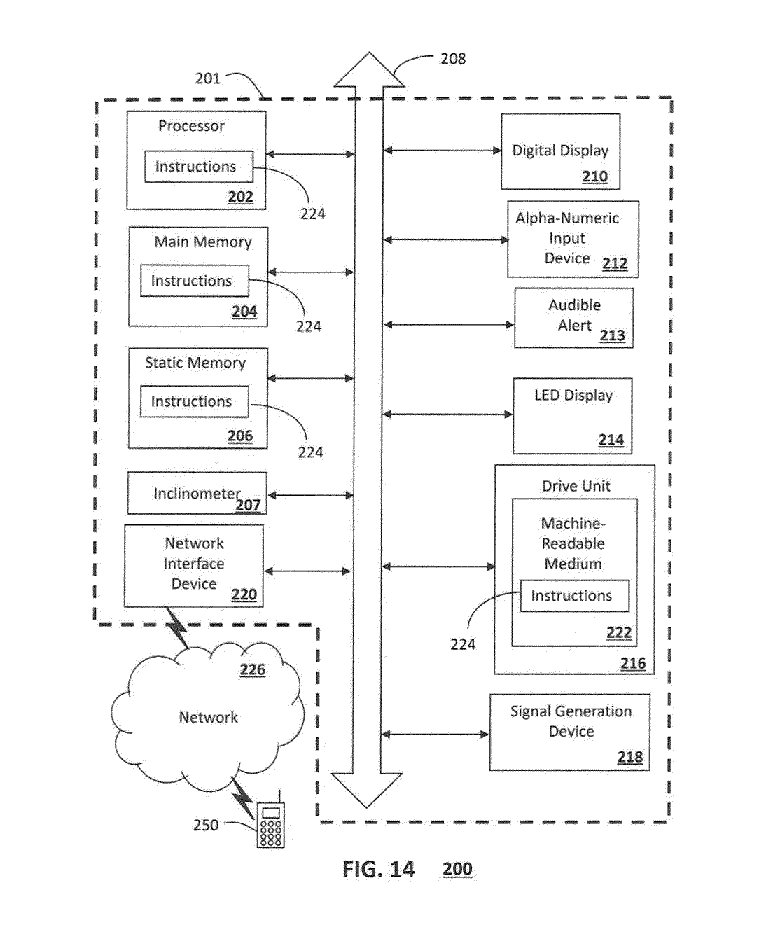

FIG. 14 depicts an exemplary diagrammatic representation of a machine in the form of a computer system 200 within which a set of instructions, when executed, may cause the machine to perform any one or more of the methodologies discussed above. In some or most embodiments herein, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment. For example, the computer system can include a recipient device 201 and a sending device 250 or vice-versa. For example, the device 201 can be a tool having the inclinometer 207 and the device 250 can be a remote computing device such as a laptop computer, smart phone, or other computer processing device which can receive and/or send signals from and/or to the device 201.

The machine may comprise a server computer, a client user computer, a personal computer (PC), a tablet PC, personal digital assistant, a cellular phone, a laptop computer, a desktop computer, a control system, a network router, switch or bridge, or any machine capable of executing a set of instructions (sequential or otherwise) that specify actions to be taken by that machine, not to mention a mobile server. It will be understood that a device of the present disclosure includes broadly any electronic device that provides voice, video or data communication. Further, while a single machine is illustrated, the term “machine” shall also be taken to include any collection of machines that individually or jointly execute a set for multiple sets) of instructions to perform any one or more of the methodologies discussed herein.

The computer system 200 and more particularly the recipient device 201 can include a controller or processor 202 (e.g., a central processing unit (CPU) graphics processing unit (GPU, or both), a main memory 204 and a static memory 206 such as DRAM, which communicate with each other via a bus 208. The computer system 200 may further include a presentation device such as a video display unit 210 (e.g. a liquid crystal display (LCD), a flat panel, a solid state display, or a cathode ray tube (CRT)). The computer system 200 may include an input device 212 (e.g., a keyboard), a cursor control device (e.g., a mouse, not shown), an audible alert device 213. a disk drive unit 216, a signal generation device 218 (e.g., a speaker or remote control that can also serve as a presentation device) and a network interface device 220. Of course, in the embodiments disclosed, many of these items are optional.

The disk drive unit 216 may include a machine-readable medium 222 on which is stored one or more sets of instructions (e.g., software 224) embodying any one or more of the methodologies or functions described herein, including those methods illustrated above. The instructions 224 may also reside, completely or at least partially, within the main memory 204, the static memory 206, and/or within the processor 202 during execution thereof by the computer system 200. The main memory 204 and the processor 202 also may constitute machine-readable media.

Dedicated hardware implementations including, but not limited to application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure. tae methods described herein are intended for operation as software programs running on a computer processor. Furthermore, software implementations can include, but are not limited to, distributed processing or component/object distributed processing, parallel processing, or virtual machine processing can also be constructed to implement the methods described herein. Further note, implementations can also include neural network implementations, and ad hoc or mesh network implementations between communication devices.

The present disclosure contemplates a machine readable medium containing instructions 224, or that which receives and executes instructions 224 from a propagated signal so that a device connected to a network environment 226 can send or receive voice, video or data, and to communicate over the network 226 using the instructions 224. The instructions 224 may further be transmitted or received over a network 226 via the network interface device 220.

While the machine-readable medium 222 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure. The terms “program,” “software application,” and the like as used herein, are defined as a sequence of instructions designed for execution on a computer system. A program, computer program, or software application may include a subroutine, a function, a procedure, an object method, an object implementation, an executable application, an applet, a servlet, a source code, an object code, a shared library/dynamic load library and/or other sequence of instructions designed for execution on a computer system. Further note that the term “integrated” or “integrated with” such as an inclinometer “integrated with” a shaft or a member can mean in some embodiments that the inclinometer is embedded within the member or shaft and in other embodiments can mean that the inclinometer is affixed to the shaft or member.

It is to be understood that the present invention can also be embodied as computer-readable codes on a computer-readable recording medium. The computer-readable recording medium is any data storage device that can store data which can thereafter be read by a computer system. Examples of the computer-readable recording medium include, but are not limited to, read-only memory (ROM), random-access memory (RAM), CD-ROMs, magnetic tapes, floppy disks, optical data storage devices, and carrier waves (such as data transmission through the Internet via wired or wireless transmission paths). The computer-readable recording medium can also be distributed over network-coupled computer systems so that the computer-readable code is stored and executed in a distributed fashion. Also, functional programs, codes, and code segments for accomplishing the present invention can be easily construed as within the scope of the invention by programmers skilled in the art to which the present invention pertains.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

While the invention has been shown and described with reference to a certain embodiment thereof, it will be understood by those skilled in the art that various changes in form and details may be made therein without departing from the spirit and scope of the invention. Consequently, the scope of the invention should not be limited to the embodiment, but should be defined by the appended claims and equivalents thereof.

What is claimed:

1. A tool, comprising;
a wrench;
an inclinometer integrated with the wrench; and
an electronic indicator operatively coupled to the inclinometer, indicating when a predetermined rotation is met or indicating when a contrary rotation direction is occurring.

2. The tool of claim 1, wherein the inclinometer is a digital inclinometer and the indicator is a digital display displaying a first display color light when a correct rotation direction is occurring and displaying a second display color light when the contrary rotation direction is occurring.

3. The tool of claim 2, wherein the first display color light is a green light and the second display color light is a red light.

4. The tool of claim 1, wherein the indicator is at least one light emitting diode that displays a plurality of different color lights.

5. The tool of claim 1, wherein the indicator is at least one light emitting diode displaying a first color light and at least a second color diode displaying a second color light.

6. The tool of claim 1, wherein the wrench is an Allen wrench.

7. The tool of claim 1, wherein the wrench is a socket wrench.

8. The tool of claim 1, wherein the inclinometer is affixed to a first member and the wrench is affixed to a second member, wherein the first member rotates relative to the second member and the inclinometer provides a measure of the relative rotation between the first and second members.

9. The tool of claim 1, wherein the wrench comprises a shaft and a power source forming a portion of the shaft.

10. The tool of claim 9, wherein the power source is a battery for powering at least one of the inclinometer or the indicator.

11. The tool of claim 1, wherein the tool adjusts threaded rods of an Ilizarov apparatus.

12. A tool, comprising:
a shaft for applying torque;
an inclinometer integrated with the shaft;
a memory storing computer instructions, the memory coupled to the inclinometer; and
a processor communicatively coupled to the memory, wherein the processor, responsive to executing the computer instructions, performs operations comprising:
generating a signal indicating when a predetermined rotation is met or when a contrary rotation direction is occurring.

13. The tool of claim 12, wherein the tool comprises an indicator operatively coupled to the inclinometer.

14. The tool of claim 12, wherein the indicator is a digital display operatively coupled to the inclinometer and wherein the computer instructions when executed by the processor performs operations comprising generating the signal indicating when the predetermined rotation is met and generating the signal when the contrary rotation direction is occurring.

15. The tool of claim 12, wherein, the indicator is a display coupled to a computing device remote from the inclinometer.

16. The tool of claim 12, comprising computer instructions, which when executed by the processor performs operations comprising generating an audible alert when the predetermined rotation is met or when the contrary rotation direction is occurring.

17. The tool of claim 12, comprising computer instructions, which when executed by the processor performs operations comprising enabling a calendaring and reminder alert function for a subsequent predetermined rotation.

18. The tool of claim 12, comprising computer instructions, which when executed by the processor performs operations comprising generating a user interface for programming the predetermined rotation and for programming a schedule for performing a number of the predetermined rotations within a predetermined time frame.

19. The tool of claim 18, further comprising a wireless interface for coupling the processor to a computing device coupled to a display.

20. A method of operating a tool, comprising:
rotating a member of the tool, the tool having an inclinometer integrated with the member;
indicating when a predetermined rotation is met by rotating the member; and
indicating when a contrary rotational direction is occurring by rotating the member in a counter-indicated direction.
